(19) United States
(12) Patent Application Publication
(10) Pub. No.: US 2026/0240574 A1
(43) Pub. Date: Aug. 20, 2026

(54) Osteosynthesis Device

(71) Applicant: Stryker European Operations Holdings LLC, Portage, MI (US)

(72) Inventors: Jacques Peyrot, Tassin La Demi Lune (FR); Tristan Meusnier, Saint-Etienne (FR); Bernard Prandi, Rennes (FR); Philippe Bellemere, Nantes (FR); Marc Augoyard, Tassin La Demi Lune (FR)

(21) Appl. No.: 19/279,688

(22) Filed: Jul. 24, 2025

Related U.S. Application Data
(63) Continuation of application No. 17/143,792, filed on Jan. 7, 2021, now Pat. No. 12,396,766.
Application No. 17/143,792 is a continuation of application No. 15/802,794, filed on Nov. 3, 2017, now Pat. No. 10,912,594.
Application No. 15/802,794 is a continuation of application No. 13/686,074, filed on Nov. 27, 2012, now Pat. No. 9,839,453.
Application No. 13/686,074 is a continuation of application No. 12/531,577, filed on Mar. 14, 2008, now Pat. No. 8,394,097.

(30) Foreign Application Priority Data
Mar. 20, 2007 (FR) 0702003

(51) Int. Cl. A61B 17/72; A61F 2/30; A61F 2/42

(52) U.S. Cl. CPC A61B 17/72; A61B 17/7266; A61B 17/7283; A61B 17/7291; A61F 2/30; A61F 2/4241; A61B 17/7225; A61F 2002/30092; A61F 2002/30166; A61F 2002/30179; A61F 2002/30563; A61F 2002/30622; A61F 2002/30841; A61F 2002/4243; A61F 2210/0014; A61F 2230/0028; A61F 2230/0058; A61F 2310/00023

(57) ABSTRACT

In one embodiment, the present invention includes an intramedullary osteosynthesis or arthrodesis implant including a central body, a first pair of legs extending from the central body to a pair of leg tips, and a second pair of legs extending from the central body, in a direction opposite the first pair of legs, to a pair of leg tips such that the central body, first pair of legs and second pair of legs are positioned alongside a longitudinal axis.

### CROSS-REFERENCE TO RELATED APPLICATIONS

This application is a continuation of U.S. application Ser. No. 17/143,792, filed Jan. 7, 2021, which is a continuation of U.S. application Ser. No. 15/802,794, filed Nov. 3, 2017, now U.S. Pat. No. 10,912,594, which is a continuation of U.S. application Ser. No. 13/686,074, filed Nov. 27, 2012, now U.S. Pat. No. 9,839,453, which is a continuation of U.S. application Ser. No. 12/531,577, filed Feb. 1, 2010, now U.S. Pat. No. 8,394,097, which is a national phase entry under 35 U.S.C. § 371 of International Application No. PCT/FR2008/050453 filed Mar. 14, 2008, published in French, which claims priority from FR0702003 filed Mar. 20, 2007, all of which are hereby incorporated herein by reference.

### FIELD OF THE INVENTION

The invention relates to the technical field of orthopaedic implants, in particular for arthrodeses and osteosyntheses.

### BACKGROUND OF THE INVENTION

It may be recalled that an osteosynthesis implant must serve to hold in place two (or more) parts of the same bone fractured or cut by a surgical operation (osteotomy), for the time necessary for the consolidation of this bone (typically 3 months).

It may be recalled that an arthrodesis is the blocking of a joint by surgery to fuse two bones into a single one, using an osteosynthesis device.

It may be recalled that the purpose of any osteosynthesis and particularly in the case of an arthrodesis is to obtain very good primary and also secondary stability, in order to obtain the consolidation in the best possible conditions, that is, in a position selected by the surgeon, by minimizing the problems of post-operative pain and edemas, by shortening the consolidation time as much as possible.

To obtain this result, the stability of the osteosynthesis associated with the implant is critical. Furthermore, the implant must also provide and maintain a slight compression on the portions of bone to be fused together, thereby facilitating this consolidation.

Various technical solutions have been proposed to carry out an arthrodesis, in particular at the tips (foot, hand, wrist, ankle, etc.).

Mention can be made, for example of basic staples which do not provide a proper fixation during consolidation, and shape memory staples which serve to place the two bone portions to be consolidated under compression, thereby corresponding to the intended purpose.

However, to obtain satisfactory stability, it is necessary to place two, or even three staples, in different planes. This significantly increases their size, thereby limiting the applications, in particular on small bones (for example in the fingers or toes).

It is also common to use extramedullary or extra-osseous plates and screws, which also require a relatively large space and cannot be used on the terminal phalanges of the fingers (distal interphalangeal arthrodesis for example). Moreover, the medium-term stability of these systems is not always guaranteed (loosening of the mounting).

Certain types of screw can be used in intramedullary cases, but in this case, the approach path requires a pulpar approach, which may generate serious complications (sepsis, etc.) and discomfort for the patient.

Use can also be made of pins which have a smaller size. However, the stability obtained is not satisfactory (problems of migration) and it is generally necessary to remove them after consolidation. With such devices, moreover, the patient cannot immerse the finger or toe treated, because the pin generally projects outside the skin.

Intramedullary osteosynthesis systems are available for long bones (tibia, femur, humerus, etc.). For example, lockable centromedullary nails are known. Apart from the fact that the locking technique is difficult, it cannot be miniaturized for extremity surgery (hand and foot).

Shape memory intramedullary devices are also available for solving part of the problems with respect to the arthrodesis or the osteosynthesis of the small fragments: for example, the solutions described in French patent 2 846 545 or French patent 2 884 406 (US 2008/0177262).

French Patent 2 846 545 describes an H-shaped device which opens in the body into an X, thanks to the use of a shape memory set around 37° C., each leg being implanted in a calibrated hole.

In practice, such a system does not allow proper introduction into the bone. This is because the preparation of 2 parallel holes in a phalange is extremely difficult due to the limited size and, above all, the parallel legs tend to open naturally during introduction and thereby exert an effect of distraction of the two fragments rather than compression.

Furthermore, the use of shape memory is very limiting due to the demands it makes on surgeons, in particular of temperature management: the implant must be fitted into the bone when cold before it warms and opens. This requires placing the implant in a support, storing it cold, and using all possible speed for implantation.

Finally, since the legs are straight, their shape memory tends to create a local support at their tips, which does not ensure satisfactory behavior and can damage the bone.

US 2008/0177262 teaches a system for easier introduction whether by the shape (eye) or by a support or a clamp that keeps the legs of the implant closed during introduction.

Nevertheless, these systems do not operate very dependably, because they do not define the optimal criteria allowing proper introduction into the bone and good anchorage: the anchoring zones always tend to open too early, thereby blocking introduction.

### OBJECT OF THE INVENTION

It is the object of the invention to remedy all these drawbacks simply, reliably, effectively and efficiently.

The problem that the invention proposes to solve is to define the success criteria for an intramedullary implant, easy to place and effective for generating primary and secondary stability of the osteosynthesis or arthrodesis focus thanks to its stiffness and its compression component.

### SUMMARY OF THE INVENTION

The inventive implant is characterized in that it comprises two bone anchoring zones on either side of a stiff stability zone, withstanding shear forces, these two anchoring zones having a possibility of high deformation at their base (in particular by elasticity) and a design such that they can adopt a closed position (in particular thanks to a suitable clamp closed at their base) for easy introduction into a calibrated centromedullary hole (prepared with an appropriate instrument), and in that owing to this particular configuration, they offer in the bone site the possibility of obtaining the final impaction without a distraction effect on the bone and sufficient expansion to ensure proper fixation in the bone.

The anchoring zones can be deformed at their base by elasticity, superelasticity or shape memory and typically consist of branches or legs, optionally connected (olive or rugby ball shape). In the open shape, these branches have an outward positive angle at their base and are curved inwardly toward their tips, whereas when the shape is closed, the angle of the base is reversed, that is negative or turned inwardly, thereby obtaining the width at the tips (impaction side) that is smaller than their base, in order to avoid impaction in the bone and blocking of the penetration of the implant.

The invention has a particularly advantageous application, which cannot however be considered as limiting, for the preparation of arthrodeses in the phalanges of the fingers and toes, especially for the proximal and distal interphalangeal joints in the hand and foot.

The device is implantable via the dorsal path (or optionally lateral or palmar/plantar), but without pulpar approach, thereby minimizing the risks of infection and improving the patient's comfort.

To take account of the anatomical features, the anchoring zones are connected to the median zone serving for strength (in particular shear) at the osteosynthesis focus by more or less long connecting zones, and the central zone may have a bend to adapt to the characteristics of the desired arthrodesis.

The material constituting the implant of the invention must allow a certain minimal opening of the anchoring zones once the implant is in the body. It may therefore be made from any sufficiently elastic implantable material such as stainless steel, titanium, a bioresorbable material such as polylactide acid PLLA.

Preferably, the implant of the invention is prepared from a shape memory material used for its property of superelasticity (or elasticity associated with the transformation of the austenite martensite phase under stress) which has the widest known elasticity range (up to 8% in elastic elongation equivalent in traction for an implant of Nitinol, a nickel-titanium alloy comprising about 55.5 to 56% by weight of nickel, the remainder being titanium).

It is also possible to use a material having a thermal shape memory around 37° C.

### BRIEF DESCRIPTION OF THE DRAWINGS

The invention is described in greater detail below in conjunction with the appended drawings in which:

FIG. 1 shows an exemplary implant of the invention in its full open position in 3 dimensions,

FIG. 2 shows the same example in its closed introduction position in its main plane,

FIG. 3 shows an exemplary implant of the invention in its full open position, in its main plane,

FIG. 4 shows an example of a bent implant according to the invention in its full open position,

FIG. 5 (5a to 5f) shows the implantation sequence: closed, half introduced still stressed, fully introduced on one side (free in the bone), and the same sequence on the other side.

### SPECIFIC DESCRIPTION

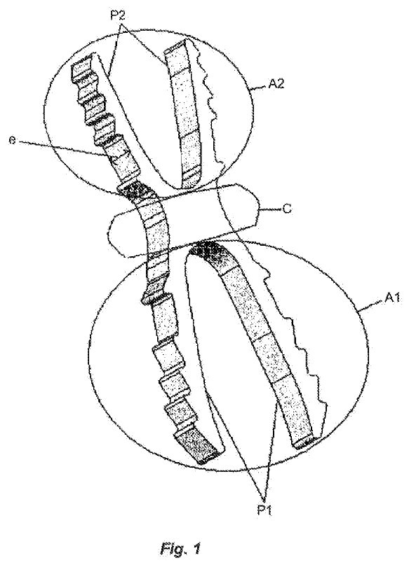

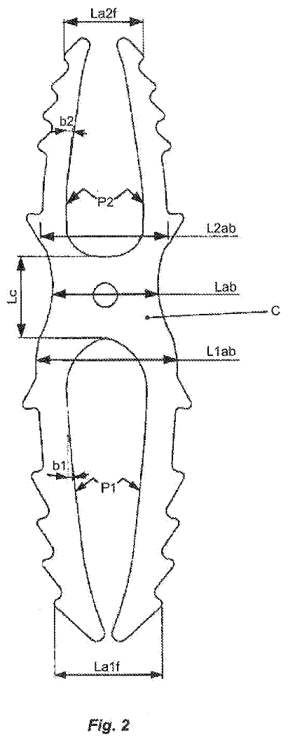

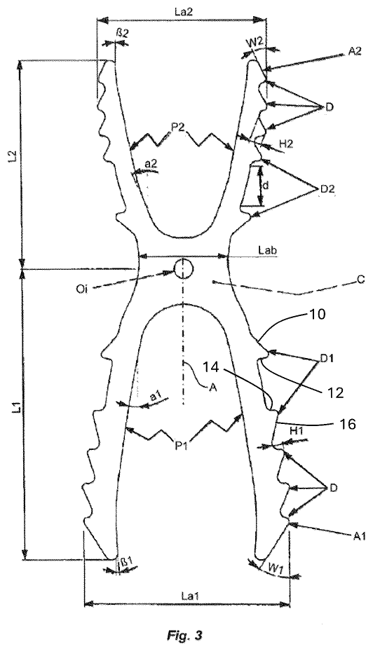

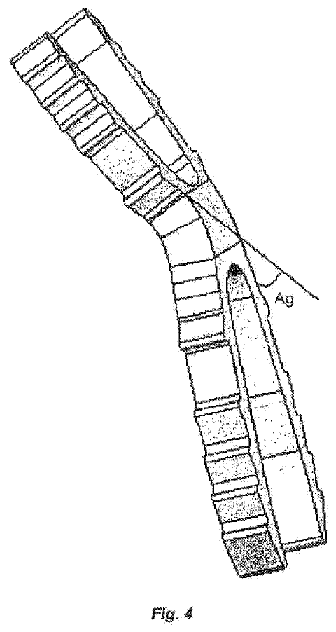

The implant is in the form of 2 anchoring zones A1 and A2 connected by a central zone C (FIG. 1) and optionally intermediate connecting zones such that in the closed position, the shape is substantially inscribed in a very elongated rectangle (FIG. 2), and in the open shape, corresponds to a wider X-shape due to the spreading of the anchoring zones A1 and A2 (FIG. 3).

The anchoring zones Al and A2 each have two legs P1 and P2 having lengths L1 and L2 (FIG. 3).

The cross-section of the implant is adapted to the implantation sites, but preferably flat in order to have good mechanical strength and reduced size (typically the thickness e is about 1 to 2 mm) (FIG. 1).

FIG. 2 shows the closed position with the various widths of the implant: Lab is the width of the central zone C, L1ab and L2ab are the widths at the base of the anchoring zones A1 and A2. These 3 widths may be equal or slightly different to adapt to the bone site. Typically, the widths are about 2 to 5 times the thickness (or 2 to 10 mm). These dimensions are adapted to the various dimensions of the hand and foot but are not limiting because they depend on the bone site of the operated patient.

The anchoring zones A1 and A2 are suitable for separation by elastic effect or by shape memory effect at their base, so that the maximum width in the open position at the tips La1 and La2 (FIG. 3) is at least equal to the width of the base of the same anchoring zone in the closed position plus 50% minimum, or plus a minimum of 1.5 mm. This means that La1>L1ab+50% or La1>L1ab+1.5 mm and that La2>L2ab+50% or La2>L2ab+1.5 mm. This opening criterion is necessary to have sufficient fixation in the bone.

As shown in FIG. 3, the legs P1 and P2 are substantially straight at their base (on about ⅓ to half of their length) and are then rounded inwardly at their tips (on about ⅓ to half of their length). In the open position, the straight inner portions of the legs P1 and P2 make positive outward angles a1 and a2 with the longitudinal implant axis A (FIG. 3), whereas in the closed position, these angles become inward negative angles b1 and b2 (FIG. 2). The upper or outer portions (toward the tip) of the legs P1 and P2 virtually undergoes no particular deformation between the two open and closed shapes.

This particular geometric arrangement ensures that in the closed position, the legs virtually touch at the tips (FIG. 2), and that the widths at the tips in the closed position La1f and La2f are lower than the widths at the respective bases L1ab and L2ab, thereby allowing easy introduction without distraction of the distal bone fragment and also obtaining the opened/closed movement by a local deformation at the base of the legs, that is by leaving the distal zone free for introducing this zone into the bone.

In order to obtain both easy introduction and sufficient opening movement, the angles a1 and a2 are preferably between +5 degrees and +25 degrees and the angles b1 and b2 between 0 degrees and −15 degrees.

Preferably, the width of the tips of the anchoring zones in the closed position La1f and La2f are lower than the widths of the bases of the zones L1ab and L2ab, minus 20%: La1f<L1ab−20% and La2f<L2ab−20%.

The legs or anchoring zones are thus “articulated” at their base, and can therefore be secured in the closed position on a support or even better a clamp, positioned at an appropriate location defined in particular in the case of an elastic material (for a shape memory material, this is not absolutely necessary since the shape does not change as long as the activation temperature is not reached), this clamp not covering more than half of the length of the legs, thereby allowing introduction of at least half of the implant into its recess.

The inside tangents at the tip of the legs P1 and P2 in the open position make angles β1 and β2 with the longitudinal axis A of the implant close to 0 degrees, in order to have a good bone contact area along the whole lengths of the legs in the open position and to prevent the bone from being touched by the tips alone (FIG. 3).

In the implant site, at body temperature, the implant can still be in the closed position, or parallel or with semiopen legs so that the force exerted by opening of the legs is transmitted to the bone and ensures proper fixation.

This “olive” arrangement of the legs, associated with an “articulation” of the base and associated with a minimal introduction of half of their length allows completion of the insertion, once the clamp has been removed.

In order to guarantee satisfactory operation, the elasticity or memory of the piece must allow a transition from the closed shape (typically width 2 to 4 mm according to the size of the site) to an open shape with a significant movement (+1.5 to +3 mm approximately).

Similarly, the force of expansion of the legs (or swelling of the olive) must be significant: typically 1 to 3 kg for an arthrodesis of the tips (force measured at 37° C. in the blocked introduction position), without being excessive: it is important for the legs to avoid opening completely and for the bone to resist so as to have a real holding force.

The legs P1 and P2 or fins may have a rough surface or even better notches D (FIG. 3) on their outer surfaces intended to be press into the spongy bone and form a good anchorage. The typical depth of these notches H1 and H2 is about 0.5 mm. The opening of the legs must be at least 1.5 times this depth in order to ensure good engagement of the notches in the bone or 1.5 mm.

The legs P1 and P2 may also have a surface covered with an osseointegration coating such as hydroxyapatite (HAP) intended to facilitate the anchorage.

To facilitate introduction into the bone, the tips of the legs P1 and P2 are bevelled with an inward angle to the longitudinal axis A of the implant W1 and W2 (FIG. 3). This angle is typically between −20 degrees and −40 degrees.

By tests on fresh cadavers and experience, an optimal level of the force was determined with a minimum allowing anchorage of the notches in the spongy bone and a maximum force to be certain to avoid damaging the implantation site.

After tests and experience, an ideal zone was found with a maximum 20% of the elastic limit of the bone measured in a blocked closed shape at 37° C., which, considering the dimensions of the implant, gives rise to maximum values of about 3 kg, and the need for a rapid lowering as soon as the anchorage is obtained, or a force divided by 2 in the semi-open position (a force of 0.5 to 1.5 kg allows good holding). In fact, if the opening force is higher than about 3 kg, introduction into the bone becomes much more difficult, or even impossible above 4 kg. Finally, in order to guarantee a damage-free site, it is necessary for the force to become negligible for a virtually complete opening. These values are indicative and depend on the arthrodesis site and the bone quality.

In one version of the invention, the notches D1 and D2 on the outside of the legs P1 and P2 allow the positioning of a clamp and introduction at the base of the legs P1 and P2 (FIG. 3). These notches are symmetrical by pairs of legs and their spacing d is the same on the legs PI and on the legs P2. A first notch D1 has a first sidewall (10) and a second sidewall (12), and a second notch D1 has a third sidewall (16) and a fourth sidewall (14) as shown in FIG. 3.

The central zone C must have a minimum length Lc equal to the length d between the notches D1 and D2 so that even in case of movement of the implant during final impaction, this zone C remains in the arthrodesis focus and performs its resistance function.

In one version of the invention, an orifice Or is provided in this central zone for positioning a holding pin to prevent migration of the implant at the time of final impaction.

As shown in FIG. 4, this central zone may be bent at an angle Ag defined between the 2 main planes formed by the legs P1 on the one hand and P2 on the other hand to adapt to the surgical requirements for adjusting the position of the arthrodesis. In most cases, the angle Ag is fixed between 0° (typically flat position for an index) and 30° (typically for a little finger).

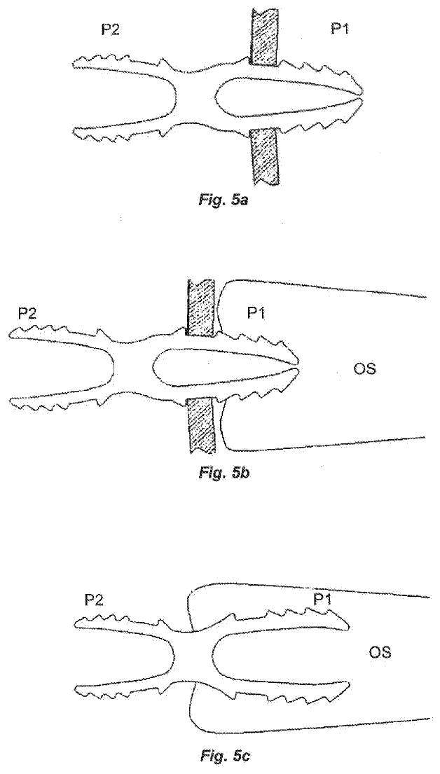

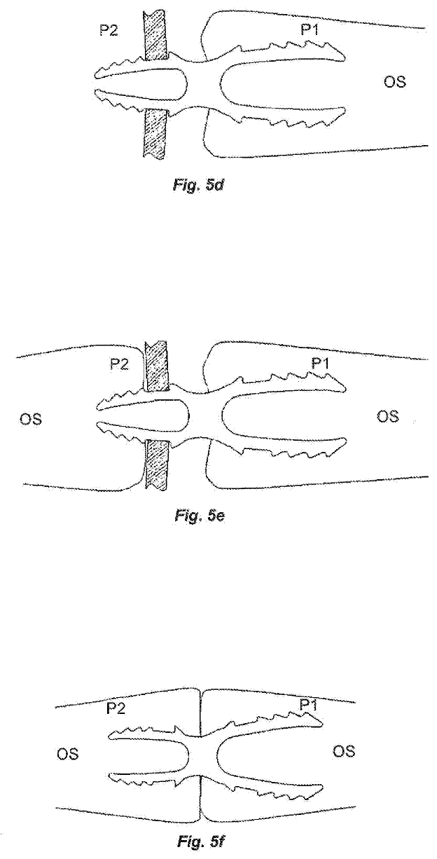

As an example, an operating technique of implantation of the inventive device for the case of an elastic or superelastic implant is described as follows as shown in FIG. 5:

### DETAILED DESCRIPTION OF THE FEATURES OF THE INVENTION AND EXEMPLARY EMBODIMENTS

Approach by dorsal path
Resection of cartilages and osteophytes
Centromedullary perforation using an appropriate instrument to make a calibrated rectangular hole having a width of substantially L1ab or L2ab and thickness of substantially e (by a suitable rasp)
Closure of the clamp side P1 (FIG. 5a)
Implant introduction side P1 to minimum half (FIG. 5b)
Clamp removal
Complete introduction side P1 (FIG. 5c)
Closure of the clamp side P2 (FIG. 5d)
Placement of the bone side P2 on the implant side P2 to about half (FIG. 5e)
Removal of the clamp
Manual impaction of the bone side P2 on the bone P1 (FIG. 5f)

In a particular embodiment, intended for a distal interphalangeal arthrodesis (hand), the implant is prepared from a superelastic Nitinol alloy (nickel—titanium in the weight proportion 55.8% nickel and 44.2% titanium).

The cross-section of the central zone C is Lab×e=2.8×1.2 mm and the legs are asymmetrical to adapt better to the shapes of the bone, minimize the implanted metal section and allow sufficient expansion for good anchorage. The length of the legs is L2=6.5 mm distal side P2 and L1=9 mm proximal side P1. The length of the central zone C is 3 mm, allowing a slight offset during closure, without affecting the shear strength. To adapt to the surgeon's choice, this central zone may be bent (typically flat or 15° or 25°).

In the closed position, the width of the proximal base L1ab is 3.8 mm and of the distal base L2ab is 3.0 mm. The opening of the legs P1 and P2 is 2.5 mm or 2.2 mm, that is La1 is 6.3 mm and La2 is 5.2 mm. In the open position the angle at the base of the legs is a1=10° and a2=22°. The straight portion is about 45% of the total length. The curvature of the distal tip of the legs is calculated so that the angle of the tangent at the tip is β1=−5° and β2=−3°. In the closed position, the angle at the base of the legs is b1=−4°, b2=−2°. And the width at tip is La1f=2.5 mm and La2f=2.1 mm.

In one embodiment of the invention, the 0.5 mm deep notches are distributed on the legs (1 notch at approximately 0.8 mm spacings).

The angle of incidence of the tip of the legs (including notches) is w1=33° and w2=24°, allowing easy introduction without the distraction effect between the two bone pieces to be osteosynthesized.

The rounded design of the anchoring zones serves to obtain a maximized contact area over the entire length in the open shape, with an impaction effect in the spongy bone, and hence a spongy packing effect.

In another example, more appropriate for arthrodesis of the thumb, the dimensions are rather the following:

Closed widths: L1ab=6.5 mm, L2ab=5 mm, with an opening of 3 to 4 mm approximately to obtain: La1=11 mm and La2=8 mm and L1=13 mm and L2=9 mm.

1. An arthrodesis implant comprising:
a rigid central zone extending along a longitudinal axis, the central zone including an orifice extending transversely through the central zone;
a first anchor zone extending from a first end of the central zone, the first anchor zone formed by a first pair of legs, each of the first pair of legs having a base portion fixed to the central zone and a tip, the legs of the first pair being deformable between a closed position for insertion into a respective bone and an open position for internally gripping the respective bone, the base portions of the first pair of legs in the open position diverging away from the central zone and in the closed position converging away from the central zone to be more closely spaced at their tips than at their base portions; and
a second anchor zone extending from a second end of the central zone opposite the first end, the second anchor zone formed by a second pair of legs, each of the second pair of legs having a base portion fixed to the central zone and a tip, the legs of the second pair being deformable between a closed position for insertion into a respective bone and an open position for internally gripping the respective bone, the base portions of the second pair of legs in the open position diverging away from the central zone and in the closed position converging away from the central zone to be more closely spaced at their tips than at their base portions.

2. The arthrodesis implant of claim 1, wherein the orifice is configured to receive a holding pin extending transversely through the central zone to prevent longitudinal migration of the arthrodesis implant within the bones.

3. The arthrodesis implant of claim 1, wherein the orifice is positioned at a midpoint of the central zone along the longitudinal axis.

4. The arthrodesis implant of claim 1, wherein the central zone has a length substantially equal to a spacing between clamp engagement notches formed on the legs of one of the anchor zones.

5. The arthrodesis implant of claim 1, wherein the orifice extends through the central zone from a first side surface to an opposite side surface thereof.

6. The arthrodesis implant of claim 1, wherein the orifice is circular in cross section.

7. The arthrodesis implant of claim 1, wherein the central zone has a flat cross-section with a thickness of about 1 to 2 mm, and the orifice extends through the thickness of the central zone.

8. The arthrodesis implant of claim 1, wherein the orifice is configured to receive a guide wire for positioning the implant during insertion.

9. The arthrodesis implant of claim 1, wherein the orifice is configured to receive a temporary fixation pin that secures the implant during final impaction of the anchor zones into the respective bones.

10. The arthrodesis implant of claim 1, wherein the orifice is located adjacent to a bend in the central zone, the bend defining an angle between the first and second anchor zones.

11. An arthrodesis implant comprising:
a rigid central zone extending along a longitudinal axis;
a first anchor zone extending from a first end of the central zone, the first anchor zone including a first pair of legs, each leg having a base portion fixed to the central zone, a tip, and an outer surface extending from the base portion to the tip, the outer surface of each leg of the first pair of legs including an osseointegration coating, the legs of the first pair being deformable between a closed position for insertion into a bone and an open position for internally gripping the bone, the base portions of the first pair of legs in the open position diverging away from the central zone and in the closed position converging away from the central zone to be more closely spaced at their tips than at their base portions; and
a second anchor zone extending from a second end of the central zone opposite the first end, the second anchor zone including a second pair of legs, each leg having a base portion fixed to the central zone, a tip, and an outer surface extending from the base portion to the tip, the outer surface of each leg of the second pair including an osseointegration coating, the legs of the second pair being deformable between a closed position for insertion into a bone and an open position for internally gripping the bone, the base portions of the second pair of legs in the open position diverging away from the central zone and in the closed position converging away from the central zone so as to be more closely spaced at their tips than at their base portions.

12. The arthrodesis implant of claim 11, wherein the osseointegration coating includes hydroxyapatite.

13. The arthrodesis implant of claim 11, wherein outer surface of each leg includes outwardly directed notches, and the osseointegration coating is disposed over at least a portion of the notches to enhance anchorage in bone.

14. The arthrodesis implant of claim 11, wherein the osseointegration coating is disposed on the outer surfaces of the legs to minimize postoperative complications by enhancing stability and reducing loosening.

15. The arthrodesis implant of claim 11, wherein the implant is made of a shape memory material, and the osseointegration coating is applied to the legs to combine elastic deformation with biological integration.

16. The arthrodesis implant of claim 11, wherein the osseointegration coating is disposed on at least a portion of the outer surface adjacent to the tips of the legs.

17. The arthrodesis implant of claim 11, wherein the osseointegration coating is disposed on substantially the entire outer surface of each leg.

18. The arthrodesis implant of claim 11, wherein the outer surface includes surface roughening beneath the osseointegration coating to enhance mechanical interlock with bone.

19. The arthrodesis implant of claim 11, wherein the osseointegration coating is configured to promote bone ingrowth into the outer surface of the legs.

20. The arthrodesis implant of claim 11, wherein the osseointegration coating is disposed only on the first anchor zone or the second anchor zone.
